(19) United States
(12) Patent Application Publication
(10) Pub. No.: US 2025/0249261 A1
(43) Pub. Date: Aug. 7, 2025

(54) PACEMAKER AND OPERATION METHOD OF SUCH PACEMAKER

(71) Applicant: BIOTRONIK SE & CO. KG, Berlin (DE)

(72) Inventor: Kurt SWENSON, Corvallis, OR (US)

(73) Assignee: BIOTRONIK SE & CO. KG, Berlin (DE)

(21) Appl. No.: 18/856,928

(22) PCT Filed: Apr. 4, 2023
(86) PCT No.: PCT/EP2023/058802

§ 371 Date: Oct. 15, 2024

Related U.S. Application Data
(60) Provisional application No. 63/335,320, filed on Apr. 27, 2022.

(30) Foreign Application Priority Data
May 20, 2022 (EP) 22174481.6

(51) Int. Cl. A61N 1/365

(52) U.S. Cl. CPC A61N 1/365

(57) ABSTRACT

A cardiac pacemaker which stays synchronized with the heart's natural cycle even if atrial sensing events are intermittently sensed and intrinsic AV conduction occurs intermittently. The pacemaker includes a processing unit, a data memory, a detector and a pacing signal generator, wherein the processing unit, the data memory, the detector and the pacing signal generator are electrically interconnected, wherein the data memory is configured to store a pre-defined programmed AV delay, wherein the detector is configured to detect an intrinsic atrial activity signal and to detect an intrinsic ventricular activity signal, wherein the processing unit is configured to produce a ventricular pace control signal and to transmit it to the pacing signal generator. A respective operation method is also disclosed.

### CROSS-REFERENCE TO RELATED APPLICATIONS

This application is the United States National Phase under 35 U.S.C. § 371 of PCT International Patent Application No. PCT/EP2023/058802, filed on Apr. 4, 2023, which claims the benefit of European Patent Application No. 22174481.6, filed on May 20, 2022 and U.S. Provisional Patent Application No. 63/335,320, filed on Apr. 27, 2022, the disclosures of which are hereby incorporated by reference herein in their entireties.

### TECHNICAL FIELD

The present invention is generally directed to a cardiac pacemaker and an operation method of such pacemaker, a respective computer program product and computer readable data carrier.

### BACKGROUND

A cardiac pacemaker (or artificial pacemaker) is a medical device that generates electrical pulses delivered by electrodes connected to or fixed at the pacemaker to cause the heart muscle chambers (i.e., the atria and/or the ventricles) to contract and therefore pump blood. By doing so this device replaces and/or regulates the function of the electrical conduction system of the heart. One purpose of a pacemaker is to maintain an adequate heart rate, either because the heart's natural pacemaker is not fast enough, or because there is a block in the heart's electrical conduction system. Additionally or alternatively, the pacemaker may stimulate different positions within the ventricles to improve their synchronization of the ventricles or provide defibrillation functions in order to treat life-threatening arrhythmias. Modern pacemakers are externally programmable and allow a health care provider (HCP) to select the optimal pacing mode(s) for individual patients.

An implantable intra-cardiac pacemaker (also known as implantable leadless pacemaker-ILP), is well known miniaturized pacemaker which is entirely implanted into a heart's ventricle (V) or atrium (A) of a patient. ILPs are considered the future of cardiac pacing. Alternative or additional functions of conventional or intra-cardiac pacemakers comprise providing other electrical or electromagnetic signals to the heart or its surrounding tissue and sensing electrical or electromagnetic signals (e.g., signals from electrical depolarization fields) or other physiological parameters of the heart and/or its surrounding tissue such as the intrinsic (i.e., the heart's natural) atrial contraction or the intrinsic (i.e., the heart's natural) ventricular contraction. Due to the highly restricted device size, an ILP has a small battery capacity. An ILP may be operated in VDD pacing mode (i.e., a pacing mode in which the ventricle is stimulated according to atrial activity and AV conduction monitoring). The VDD mode is suitable for patients with AV conduction disorders, of which there are several types. In particular, this present invention pertains to patients with intermittent AV conduction.

In the VDD mode, the pacemaker synchronizes ventricular pacing with the intrinsic atrial timing by sensing when atrial contractions occur. In an ILP that is implanted in the right ventricle, the atrial contraction information can be detected as a far field signal, but with less reliability and accuracy than in a dual chamber pacemaker where there is a lead in the right atrium as well as the right ventricle. In the VDD mode, since there are no atrial paces, the pacemaker is completely reliant on synchronizing to the cardiac conditions rather than being able to control the timing in both chambers as can be done in the DDD pacing mode (in which the atrium and ventricle are paced). This requires the pacemaker to learn both the cardiac cycle rate and also to determine when in the cycle the atrial sense event (i.e., the natural atrial contraction) occurs. The cardiac cycle interval (which is the inverse of the heart rate) is composed of the AV portion (portion of the cardiac cycle between an atrial and the following ventricular sense event) and the VA portion (portion of the cardiac cycle between a ventricular and the following atrial sense event). The AV portion of the cardiac cycle is also called AV delay in the following. The ventricular sense event is a sensed natural ventricular contraction. In the VDD mode the pacing of the ventricle may be based on a pre-programmed AV delay or an adapted AV delay (e.g., adapted according to the patient's sporting activity level or heart rate).

In traditional VDD timing, the presence of a ventricular sense event (short: Vs) is often intermittent. In an ILP, the atrial sense event (short: As) can also be intermittent, in particular due to far-field detection of atrial signals (e.g., detection of electrical depolarization fields which are generated in heart's tissue which is not immediately adjacent to the electrode). Learning the intrinsic rate always requires sensing in either the atrium or the ventricle or both. Furthermore, sensing must occur in at least two consecutive cardiac cycles, so that the cycle interval can be measured. The most straight forward way to make the interval measurement is to use a counter to count a regular known clock rate, and to start the count on every ventricular event, either a ventricular sense event or a ventricular pace (Vp). In a cycle which ends in a ventricular sense event, the cycle interval is the between Vx and Vs (VxVs) elapsed duration (Vx means one of Vp and Vs). In a cycle which has an atrial sense (As), the atrial sense event starts an AV delay. If the cycle ends in a ventricular sense, the cycle interval time is the same as the first case. If the cycle ends in a ventricular pace, the cycle interval time is the VxAsVp elapsed duration. If the cycle has neither a ventricular nor an atrial sense event and has started with a ventricular pacing the cycle cannot be used to measure the intrinsic (i.e., natural) heart rate which may change during the day, for example, according to the patient's activity level.

The pacing functionality of a conventional or intra-cardiac pacemaker is aimed at staying synchronized with the heart's natural activity during such cycles. When the programmed AV delay is different from the intrinsic AV conduction time and with intermittent sensing atrial events and intermittent AV conduction, the measurement of the cardiac rate may lead to errors in filling in the timing in which no senses are present. This may lead to loss of synchronization between the pacemaker and the heart.

In an ILP that is implanted in the right ventricle, the atrial contraction information can be detected as a far field signal in the ventricular sense signal. Because the ventricular artifacts in the signal are so much larger, due to being near field, than the atrial artifacts, it is challenging to discriminate the atrial components so that atrial contractions can be detected. In an AV synchronized heart, the atria and the ventricles are active at different time periods, so this can be used as a means to aid in this discrimination. Ideally, the atrial contraction is occurring after the ventricular T-wave (repolarization artifact). A pacemaker can determine when to start looking for atrial artifacts based on when the ventricular event occurs and under what conditions. Since the cardiac interval is divided into the VA part and the AV part, and because when the pacemaker paces in the ventricle it can determine the time relationship between the atrial contractions that it detects and the ventricular events, this can be used to increase the probability of detecting atrial contractions.

Accordingly, there is the need for a cardiac pacemaker which stays synchronized with the heart's natural cycle even if atrial sensing events are intermittently sensed and intrinsic AV conduction occurs intermittently, as well. Similarly, a corresponding operation method of such pacemaker is needed.

The present disclosure is directed toward overcoming one or more of the above-mentioned problems, though not necessarily limited to embodiments that do.

### SUMMARY

At least the above problem is solved with a cardiac pacemaker comprising the features of claim 1 and an operation method comprising the features of claim 5.

In particular, at least the above problem is solved by a cardiac pacemaker comprising a processing unit, a data memory, a detector and a pacing signal generator, wherein the processing unit (processor), the data memory, the detector and the pacing signal generator are electrically interconnected. The data memory is configured to store a pre-defined programmed AV delay, wherein the detector is configured to detect an intrinsic atrial activity signal and to detect an intrinsic ventricular activity signal. The processing unit is configured to produce a ventricular pace control signal and to transmit it to the pacing signal generator, wherein the processing unit is further configured to determine an atrial sense event from the intrinsic atrial activity signal received from the detector and an intrinsic AV delay from the atrial sense event and the subsequent intrinsic ventricular activity signal received from the detector. Furthermore, the processing unit is configured to determine continuously an average intrinsic AV delay from the most recently determined intrinsic AV delay and previously determined AV delays and to store the average intrinsic AV delay in the data memory. In one embodiment, the average intrinsic AV delay determined in earlier cycles and the most recently measured intrinsic AV delay are used to calculate a new (actual) average intrinsic AV delay value. If no intrinsic AV delay is determined in the actual cycle, the average intrinsic AV delay is not changed. The processing unit is further configured to produce the ventricular pace control signal after one atrial sense event is determined based on the (actual) average intrinsic AV delay if the average intrinsic AV delay is fresh or based on the programmed AV delay or on a differently determined AV delay if the average intrinsic AV delay is stale. In other words, the average intrinsic AV delay embodying the heart's natural AV delay is used for ventricular pacing only if it is frequently (i.e., with a reasonable number) measured in a recent time period. If this condition is not true, which means that the average intrinsic AV delay is stale, i.e., it is seldom measured and/or was measured before a longer time, the ventricular pace control signal is determined based on the programmed AV delay or a differently determined AV delay. The differently determined AV delay may be an AV delay adapted based on another physiological condition of the patient, for example, the patient's heart rate. This rate may be based on using an accelerometer integrated within the pacemaker, or it may be based on the heart's intrinsic rhythm. For example, three different AV delay values may be stored in the data memory, one for low heart rate, one for medium heart rate and one for high heart. The AV delay value corresponding to the measured heart rate may be used as AV delay for the ventricular pacing if the average intrinsic AV delay is stale. This rate-based AV delay is a well-known feature of pacemakers, called dynamic AV delay. The conversion from rate to AV interval may also be calculated by the pacemaker rather than using a look-up from a table.

The atrioventricular delay, also called AV delay, is the time interval between the beginning of atrial systole and the beginning of the ventricular systole.

The above cardiac pacemaker provides a better synchronization of the cardiac pacing and the heart's natural cycle. The cardiac pacemaker may be an ILP or a (conventional) pacemaker whose device is not located within the patient's heart.

The processing unit processes signal data received from the detector, for example, intrinsic atrial activity signals or intrinsic ventricular activity signals. From these signals, the processing unit may derive an atrial sense event or a ventricular sense event based on different known signal assessment methods such as high pass filter, low pass filter or band pass filter. The terms “atrial sense event” and “ventricular sense event” mean the respective natural signals referring to a natural contraction of the respective atrium and ventricle. For example, if the signal data received from the detector is an IEGM (intracardiac electrogram), the processing unit may assess this IEGM. The atrial sense event may be derived from the IEGM if a P-wave is identified and the ventricular sense event if a QRS-complex or R-wave is detected in the IEGM. From atrial sense events and subsequent ventricular sense events the processing unit determines an intrinsic AV delay. The AV delay is the time period between an atrial sense event and the next (subsequent) ventricular sense event, wherein in one embodiment the time period cannot be longer than a predefined AV delay threshold. The AV delay threshold is defined and stored in the data memory in order to exclude cases in which after one atrial sense event the following ventricular sense event and atrial sense event of the next cycle are not detected. With regard to the present invention the processing unit is generally regarded as a functional unit of the pacemaker, that interprets and executes instructions comprising an instruction control unit and an arithmetic and logic unit. The processing unit may comprise a microprocessor, a controller, a digital signal processor (DSP), an application specific integrated circuit (ASIC), a field-programmable gate array (FPGA), discrete logic circuitry or any combination thereof. Further, the processing unit may comprise a counter and a clock. The counter may be used to count clock signals of the clock. The counter may be started at each sensed atrial event and count the number of clock signals until a ventricular sense event is determined or a ventricular pace signal is provided by the pacing signal generator. The intrinsic AV delay may be determined from the number of counted clock signals between the atrial sense event and the subsequent ventricular sense event (within one cycle). In particular, the processing unit is configured to determine continuously the average intrinsic AV delay and the ventricular pace control signal. Based on the ventricular pace control signal the pacing signal generator produces the electrical pacing signal(s) in order to transfer it to the electrodes which apply the signal(s) to the heart's tissue adjacent to the electrode.

The data memory of the pacemaker may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device.

The detector may comprise means to detect IEGM signals as indicated above. Additionally or alternatively, the detector may comprise an accelerometer, a vibration sensor, an acoustic sensor (including ultrasound), an impedance sensor, a pressure sensor and/or any other mechanical, electric and/or magnetic sensor that is capable to detect the cardiac activity, in particular to detect the atrial or ventricular activity of the patient's heart, i.e., information on contraction of the heart's chambers in order to determine an atrial sense event, a ventricular sense event and an intrinsic AV delay, for example, an “atrial kick” occurring during contraction of the atrium. The detector collects the activity signals of the heart and transform them into electrical signals. Further, the detector may digitize analog signals or smooth them. Some pre-processing steps may be provided by the detector, as well. The signals produced by the detector may be transmitted to the processing unit directly or after a pre-defined time delay.

The pacing signal generator produces the pacing signals which are then applied via the electrodes to the heart's tissue. The pacing signals are pulses that begin at a desired time point and have a desired intensity and length. Further, the pulse form may be varied. Information on the pacing signals that are necessary to produce the correct pacing signals are provided by the pace control signal of processing unit.

The pacemaker may comprise further modules such as a communication unit for communication with a remote computer and a power supply such as a battery. The communication unit may exchange messages with the external (at least partially extracorporeally) remote computer, for example, in one single direction or bidirectionally. The communication may be provided wirelessly via the patient's body and/or the air using electromagnetic waves, for example, Bluetooth, WLAN, ZigBee, NFC, Wibree or WiMAX in the radio frequency region, or IrDA or free-space optical communication (FSO) in the infrared or optical frequency region or by wire (electrical and/or optical communication). The remote computer is a functional unit that can perform substantial computations, including numerous arithmetic operations and logic operations without human intervention, such as, for example, a personal mobile device (PMD), a desktop computer, a server computer, clusters/warehouse scale computer or embedded system. The pacemaker's units and components may be contained within a hermetically sealed housing.

In one embodiment the pacemaker comprises electrodes for application of an electrical pacing signal provided by the pacing signal generator. The electrodes are electrically connected to the pacing signal generator via a header of the pacemaker. In one embodiment the electrode may comprise a lead which may be detachable connected to the respective connector at the header. With regard to an ILP one electrode may be located at a distal end of the ILP, close to a fixation member by which the ILP is fixed in the tissue of the patient's heart, for example, within the inner tissue of a ventricle. A second electrode may be located at the proximal end of the ILP or a part of the ILP housing that may, for example, serve as counter electrode. Further, the electrodes may be adapted to detect intrinsic ventricular activity signals or intrinsic atrial activity signals by picking up electrical potentials. The electrodes may thereby be part of the detector of the pacemaker.

In one embodiment, the average intrinsic AV delay is determined from the most recently determined intrinsic AV delay and previously determined AV delays using a recursive filter. The intent of the filtering is to minimize the influence of outlier timing from the measurement average. Moving average, weighted average or other well-known averaging techniques could also be used to achieve this. Intrinsic AV delays can naturally shift, for example, as a result of heart rate, and the time constants used to do the average would be selected to allow following of such shifts with minimal delay while still minimizing the effect of, for example, ectopic events in the heart. A recursive filter that weighs the new inputs by ¼ would, for example, be suitable.

In one embodiment, the processing unit is configured to determine a freshness counter value which is carried on from one cycle to the next cycle and is increased by a first pre-determined value after one intrinsic AV delay is determined and is decreased by a second pre-determined value after one ventricular pace control signal is transmitted to the pacing signal generator. The first value and the second value may be the same or different. In each cycle, the freshness counter is value checked and the actual average intrinsic AV delay is considered fresh if the freshness counter value is equal to or above a freshness threshold and the actual average intrinsic AV delay is considered stale if the freshness counter value is below the freshness threshold. This is a simple and effective way of determining whether the average intrinsic AV delay is fresh or stale. It may be realized in hard- or software. The initial value of the freshness counter (for example, to be used after a restart or the pacemaker) is, for example, 0, indicating that there is no recent history so the AV average should be considered stale. The first pre-determined value may be, for example, 1 and the second pre-determined value, for example, also 1. The freshness threshold may be chosen, for example, 4, indicating that several AV intervals have been measured in the last half dozen cycles. An alternative to using such an up/down counter approach is to use a ‘one-shot’ mechanism, wherein a counter is set to a maximum value, for example, 7, every time an AV interval is measured, and to decrement the counter, for example, by 1, every time a ventricular pace is delivered (down to a minimum of 0). As long as the counter remains at a value greater than 0, the average AV interval measurement is considered fresh. Otherwise it is considered stale. Other means determining whether the average intrinsic AV delay represents current values may be used, as well. For example, each determined intrinsic AV delay may be provided with a time stamp. The time stamp may then be used to determine the most recent values (e.g., within a pre-defined time period) from which an average value may be calculated (e.g., an arithmetic mean, geometric mean, harmonic mean or median value).

In one embodiment, in case the average intrinsic delay is stale, the ventricular pace control signal is produced by the processing unit after one atrial sense event is determined based on a pre-defined hysteresis value and the programmed AV delay or the differently determined AV delay, wherein the programmed AV delay is extended by the hysteresis value or the differently determined AV delay is extended by the hysteresis value. This embodiment realizes a hysteresis feature that is used to encourage ventricular sensing over pacing. By this embodiment the time before the ventricular pace is delivered is extended to allow an AV conduction that is in progress to reach the ventricle first, causing a ventricular sense event that inhibits the ventricular pacing. In cycles in which the average intrinsic AV delay is used instead of the programmed AV delay (i.e., in cycles in which the average intrinsic AV delay is considered fresh), this hysteresis feature is skipped. While this will result in more ventricular paces than would occur with the hysteresis feature active, the hysteresis extension alters the pacing rate (lengthens it) which may contribute to loss of AV synchrony. It is unknown whether AV synchrony, which helps preserve tricuspid and mitral valve functionality, is more important in long term heart health than encouraging the use of intrinsic AV conduction (i.e., avoiding ventricular pacing), which helps encourage RV/LV synchronization.

At least the above problem is further solved by an operation method of a cardiac pacemaker, wherein the pacemaker comprises a processing unit, a data memory, a detector and a pacing signal generator, wherein the processing unit, the data memory, the detector and the pacing signal generator are electrically interconnected, wherein the data memory stores a pre-defined programmed AV delay, wherein the detector detects an intrinsic atrial activity signal and an intrinsic ventricular activity signal, wherein the processing unit produces a ventricular pace control signal and transmits it to the pacing signal generator, wherein the processing unit determines an atrial sense event from the intrinsic atrial activity signal received from the detector and an intrinsic AV delay from the atrial sense event and the subsequent intrinsic ventricular activity signal received from the detector and determines continuously an average intrinsic AV delay from the most recently determined intrinsic AV delay and previously determined AV delays and stores the average intrinsic AV delay in the data memory, wherein the processing unit produces the ventricular pace control signal after one atrial sense event is determined based on the average intrinsic AV delay if the average intrinsic AV delay is fresh or based on the programmed AV delay or on a differently determined AV delay if the average intrinsic AV delay is stale. The above method has the advantages indicated for the respective pacemaker above. In one embodiment, the average intrinsic AV delay is determined from the most recently determined intrinsic AV delay and previously determined AV delays using a recursive filter.

In one embodiment, the processing unit determines a freshness counter value which is increased by a first pre-determined value after one intrinsic AV delay is determined and is decreased by a second pre-determined value after one ventricular pace control signal is transmitted to the pacing signal generator, wherein the average intrinsic AV delay is considered fresh if the freshness counter value is equal to or above a freshness threshold and is considered stale if the freshness counter value is below the freshness threshold.

In one embodiment, in case the average intrinsic delay is stale, the ventricular pace control signal is produced after one atrial sense event is determined based on a pre-defined hysteresis value and the programmed AV delay or the differently determined AV delay, wherein the programmed AV delay is extended by the hysteresis value or the differently determined AV delay is extended by the hysteresis value.

The above embodiments of the operation method have the same advantages as the above pacemaker. Embodiments of the pacemaker indicated above may be realized in the operation method analogously. It is referred to the above explanation of the pacemaker in this regard.

The above method is, for example, realized as a computer program which comprises instructions which, when executed, cause the processing unit (processor) to perform the steps of the above method (to be executed by the medical device, in particular at its processor) which is a combination of above and below specified computer instructions and data definitions that enable computer hardware to perform computational or control functions or which is a syntactic unit that conforms to the rules of a particular programming language and that is composed of declarations and statements or instructions needed for a above and below specified function, task, or problem solution.

Furthermore, a computer program product is disclosed comprising instructions which, when executed by the processing unit, cause the processing unit to perform the steps of the above defined method. Accordingly, a computer readable data carrier storing such computer program product is disclosed.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawing, wherein:

FIG. 1 shows a first embodiment of the pacemaker within a cross section of a patient's heart, and

FIG. 2 depicts a functional block diagram of the pacemaker shown in FIG. 1.

### DETAILED DESCRIPTION

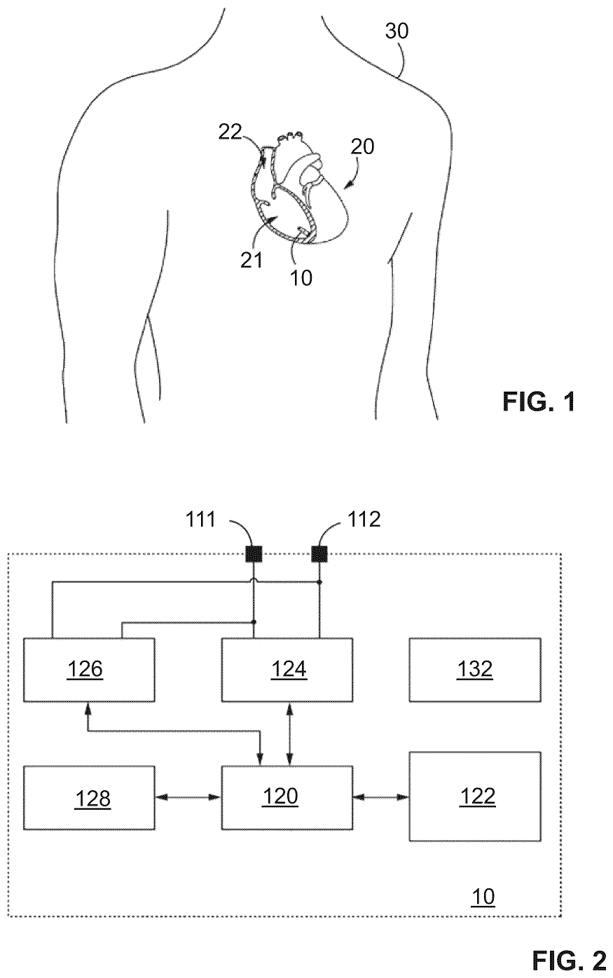

FIG. 1 shows an example leadless ventricular pacemaker (ILP) 10 implanted within the heart 20 of a patient 30. ILP 10 may be configured to be implanted within the right ventricle 21 of the heart 20 and pace this ventricle, sense intrinsic ventricular depolarizations and depolarizations of the atria (e.g., the right atrium 22), and inhibit ventricular pacing in response to detected ventricular depolarization. A programmer (not shown) may be used to program ILP 10 and retrieve data from ILP 10. The ILP 10 is one example of a cardiac pacemaker 10. Other embodiments of the cardiac pacemaker 10 are possible.

FIG. 2 shows a functional block diagram of the ILP 10 configured for implantation within ventricle 21 (FIG. 1). The ILP 10 comprises a processing unit 120 with a clock and at least one counter for the clock signals, a data memory 122, a pacing signal generator 124, a detector 126, a communication unit 128, and a power source 132. The power source 132 may include a battery, e.g., a rechargeable or non-rechargeable battery. The power source 132 provides electrical energy to all units and components of the ILP 10, in particular to all units mentioned above and is therefore electrically connected to these units and components. Units included in ILP 10 represent their respective functionality. Similar or identical units and functionality may also be included in the ILP 10. Units of the present disclosure may include any discrete and/or integrated electronic circuit components that implement analog and/or digital circuits capable of producing the functions attributed to the units herein. For example, the units may include analog circuits, e.g., amplification circuits, filtering circuits, and/or other signal conditioning circuits. The units may also include digital circuits, e.g., combinational or sequential logic circuits, memory devices, etc. The data memory 122 may include any volatile, non-volatile, magnetic, or electrical media mentioned above. Furthermore, the processing unit 120 may include instructions that, when executed by one or more processing circuits, cause the units to perform various functions attributed to these units herein. The functions attributed to the units herein may be embodied as one or more processors, hardware, firmware, software, or any combination thereof. Depiction of different features as units is intended to highlight different functional aspects, and does not necessarily imply that such units must be realized by separate hardware or software components. Rather, functionality associated with one or more units may be performed by separate hardware or software components, or integrated within common or separate hardware or software components. Data memory 122 may include computer-readable instructions that, when executed by processing unit 120, cause processing unit 120 to perform the various functions attributed to processing unit 120 herein. Further, data memory 122 may store parameters for these functions, e.g., pacing signal parameters. For example, data memory 122 may store a pre-defined programmable AV delay. The pacing instructions and pacing signal parameters may be updated by the programmer using the communication unit 128. The communication unit 128 may comprise an antenna or a transceiver.

The processing unit 120 may communicate with pacing signal generator 124 and detector 126 thereby transmitting signals. Pacing signal generator 124 and detector 126 are electrically coupled to electrodes 111, 112 of the ILP 10. Detector 126 is configured to monitor signals from electrodes 111, 112 in order to monitor electrical activity of heart 20. Further, the detector 126 may include an accelerometer, an acoustic sensor and/or a pressure sensor. Pacing signal generator 124 is configured to deliver electrical stimulation signals to ventricle 21 via electrodes 111, 112.

Processing unit 120 may control pacing signal generator 124 to generate and deliver electrical stimulation to ventricle 21 via electrodes 111, 112. Electrical stimulation may include pacing pulses. Processing unit 120 may control pacing signal generator 124 to deliver electrical stimulation therapy according to one or more therapy programs including pacing parameters, which may be stored in data memory 122.

Detector 126 may include circuits that acquire electrical signals (e.g., electric depolarization signals) from the heart including intrinsic cardiac electrical activity, such as intrinsic ventricular activity and/or intrinsic ventricular activity. Detector 126 may filter, amplify, and digitize the acquired electrical signals to generate raw digital data. Processing unit 120 may receive the digitized data generated by detector 126.

Processing unit 120 may assess the raw digital data received from the detector 126 and is configured to identify atrial sense events and ventricular sense events. For example, static or dynamic amplitude threshold detectors may be used to distinguish events, timing patterns may be used to distinguish senses from noise, and events recognized as ventricular sense signals or ventricular repolarization (T-wave) signals may be used to define a time period used to qualify potential atrial sense signals.

ILP 10 may include a housing, fixation tines, and the electrodes 111, 112. The housing may have a pill-shaped cylindrical form factor in some examples. Fixation tines are configured to connect (e.g., anchor) ILP 10 to heart 20. Fixation tines may be fabricated from a shape memory material, such as Nitinol. In some examples, fixation tines may connect ILP 10 to heart 20 within one of the chambers of heart 20. For example, as illustrated and described herein with respect to FIG. 1, fixation tines may be configured to anchor ILP 10 to heart 20 within right ventricle 21. Although ILP 10 includes a plurality of fixation tines that are configured to anchor ILP 10 to cardiac tissue in the right ventricle, it is contemplated that a pacemaker according to the present disclosure may be fixed to cardiac tissue in other chambers of a patient's heart 20 using other types of fixation mechanisms.

ILP 10 may include two electrodes 111, 112, although more than two electrodes may be included on a pacemaker in other examples. Electrodes 111, 112 may be spaced apart a sufficient distance to be able to detect various electrical signals generated by the heart 20, such as P-waves generated by atria and QRS complex generated by ventricles. The housing houses electronic components of ILP 10. Electronic components may include any discrete and/or integrated electronic circuit components that implement analog and/or digital circuits capable of producing the functions attributed to ILP 10 described above.

The communication unit 128 may enable ILP 10 to communicate with other electronic devices, such as a programmer or other external patient monitor. In some examples, the housing may house an antenna for wireless communication. Housing may also include the power source 132.

The processing unit 120 may be adapted to control pacing of the right ventricle 21 using the known VDD mode. The counter of the processing unit 120 used to time the AV delay (for providing the ventricular pace signal) may also be used to measure intrinsic AV delays. This timer is restarted if one atrial sense event is detected by the processing unit 120 within the activity signals derived from the detector 126. If no ventricular sense event is seen before it times out, a ventricular pace control signal is produced by the processing unit 120 and transmitted to the pacing signal generator 124. In the case where a ventricular sense event is seen, the counter value at the instant of the sense detection is a measurement of the intrinsic AV delay. This value may be averaged over several cycles by weighting the current measurement value using arithmetic logic and by weighting a stored running average value by using arithmetic logic and summing them with a logical adder and dividing them by logically shifting to give a weighted average, so that the output represents the most recent average of the intrinsic AV delays, called average intrinsic AV delay. A ‘one-shot’ mechanism (could be either hardware or software) can be used to keep track of whether the average intrinsic AV delay average is fresh enough to use or not, for example, a freshness counter. Directly after restart of the ILP 10 the freshness counter has a pre-defined value, for example, 0. Further, the freshness counter has a maximum value of 7 and can therefore take values between 0 and 7. The freshness counter is loaded with a pre-defined first value (e.g., 7) every time an AV conduction is detected (i.e., every time a ventricular sense event is detected after one atrial sense event of the same cycle). The value is decremented every time a ventricular pace is delivered by a pre-defined second value (e.g., 1), that is for every cycle in which there is no AV conduction to measure. If the count of the freshness counter gets down to a pre-defined freshness-threshold (e.g., 0), the average intrinsic AV delay is considered stale. If the freshness counter is greater than 0, the average intrinsic AV delay is considered fresh.

In a cycle in which an atrial sense event is detected by the processing unit 120, if the determined average intrinsic AV delay is fresh, the AV timer is used to time its duration. If no ventricular sense event is seen before it times out, a ventricular pace control signal is produced by the processing unit 120, transmitted to the pace signal generator 124 and a respective pace signal is delivered by the electrodes 111, 112 to the patient's heart 20. If the average intrinsic AV delay is stale, the user programmed AV delay is used instead which is stored in the data memory 122.

In one embodiment, a hysteresis feature is used to encourage ventricular sensing over pacing. The time before the ventricular pace is delivered is extended by a pre-defined hysteresis value to allow an AV conduction that is in progress to reach the ventricle first, causing a ventricular sense event that inhibits the ventricular pacing. The pre-defined hysteresis value may be stored in the data memory 122 and programmed by the programmer. However, in cycles in which the average intrinsic AV delay is used instead of the programmed AV delay, the hysteresis feature is skipped.

In one embodiment, the above algorithm may be configured as an optional function of the ILP 10. If the user programs, for example, “AV Persist” on, this algorithm would be used. If the user programs, for example, “AV Persist” off, the programmed AV delay would always be used. This allows the physician to decide whether to prioritize intrinsic AV conduction times or minimization of ventricular pacing.

The above ILP 10 and explained method uses an AV delay (i.e., the average intrinsic AV delay) that more accurately represents the normal AV conduction time when that information is available. It alternatively uses the user programmed AV delay when measurements are not fresh enough to be meaningful, i.e., during periods of loss of AV conduction. Thereby the ability of VDD in the ILP 10 is improved to stay in synchronization with the natural cardiac timing of the patient's heart 20. Further, alternating long and short cardiac cycles are reduced when the intrinsic and programmed AV delays are different and AV conduction is following a 2:1 pattern.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A cardiac pacemaker comprising a processing unit, a data memory, a detector and a pacing signal generator, wherein the processing unit, the data memory, the detector and the pacing signal generator are electrically interconnected, wherein the data memory is configured to store a pre-defined programmed AV delay, wherein the detector is configured to detect an intrinsic atrial activity signal and to detect an intrinsic ventricular activity signal, wherein the processing unit is configured to produce a ventricular pace control signal and to transmit it to the pacing signal generator, wherein the processing unit is further configured to determine an atrial sense event from the intrinsic atrial activity signal received from the detector and an intrinsic AV delay from the atrial sense event and the subsequent intrinsic ventricular activity signal received from the detector and to determine continuously an average intrinsic AV delay from the most recently determined intrinsic AV delay and previously determined AV delays and to store the average intrinsic AV delay in the data memory, wherein the processing unit is further configured produce the ventricular pace control signal after one atrial sense event is determined based on the average intrinsic AV delay if the average intrinsic AV delay is fresh or based on the programmed AV delay or on a differently determined AV delay if the average intrinsic AV delay is stale.

2. The pacemaker according to claim 1, wherein the average intrinsic AV delay is determined from the most recently determined intrinsic AV delay and previously determined AV delays using a digital averaging.

3. The pacemaker according to claim 1, wherein the processing unit is configured to determine a freshness counter value which is increased by a first pre-determined value after one intrinsic AV delay is determined and is decreased by a second pre-determined value after one ventricular pace control signal is transmitted to the pacing signal generator, wherein the average intrinsic AV delay is considered fresh if the freshness counter value is equal to or above a freshness threshold and is considered stale if the freshness counter value is below the freshness threshold.

4. The pacemaker according to claim 1, wherein in case the average intrinsic delay is stale, the ventricular pace control signal is produced after one atrial sense event is determined based on a pre-defined hysteresis value and the programmed AV delay or the differently determined AV delay, wherein the programmed AV delay is extended by the hysteresis value or the differently determined AV delay is extended by the hysteresis value.

5. An operation method of a cardiac pacemaker, wherein the pacemaker comprises a processing unit, a data memory, a detector and a pacing signal generator, wherein the processing unit, the data memory, the detector and the pacing signal generator are electrically interconnected, wherein the data memory stores a pre-defined programmed AV delay, wherein the detector detects an intrinsic atrial activity signal and an intrinsic ventricular activity signal, wherein the processing unit produces a ventricular pace control signal and transmits it to the pacing signal generator, wherein the processing unit determines an atrial sense event from the intrinsic atrial activity signal received from the detector and an intrinsic AV delay from the atrial sense event and the subsequent intrinsic ventricular activity signal received from the detector and determines continuously an average intrinsic AV delay from the most recently determined intrinsic AV delay and previously determined AV delays and stores the average intrinsic AV delay in the data memory, wherein the processing unit produces the ventricular pace control signal after one atrial sense event is determined based on the average intrinsic AV delay if the average intrinsic AV delay is fresh or based on the programmed AV delay or on a differently determined AV delay if the average intrinsic AV delay is stale.

6. The method of claim 5, wherein the average intrinsic AV delay is determined from the most recently determined intrinsic AV delay and previously determined AV delays using digital averaging.

7. The method of claim 5, wherein the processing unit determines a freshness counter value which is increased by a first pre-determined value after one intrinsic AV delay is determined and is decreased by a second pre-determined value after one ventricular pace control signal is transmitted to the pacing signal generator, wherein the average intrinsic AV delay is considered fresh if the freshness counter value is equal to or above a freshness threshold and is considered stale if the freshness counter value is below the freshness threshold.

8. The method of claim 5, wherein in case the average intrinsic delay is stale, the ventricular pace control signal is produced after one atrial sense event is determined based on a pre-defined hysteresis value and the programmed AV delay or the differently determined AV delay, wherein the programmed AV delay is extended by the hysteresis value or the differently determined AV delay is extended by the hysteresis value.

9. A computer program product comprising instructions which, when executed by a processing unit, cause the processing unit to perform the steps of the method according to claim 5.

10. Computer readable data carrier storing a computer program product according to claim 9.
